(19) United States
(12) Patent Application Publication
(10) Pub. No.: US 2021/0145823 A1
(43) Pub. Date: May 20, 2021

(54) CRENOLANIB FOR TREATING FLT3 MUTATED PROLIFERATIVE DISORDERS ASSOCIATED MUTATIONS

(71) Applicant: Arog Pharmaceuticals, Inc., Plano, TX (US)

(72) Inventor: Vinay K. Jain, Dallas, TX (US)

(21) Appl. No.: 17/159,649

(22) Filed: Jan. 27, 2021

Related U.S. Application Data
(63) Continuation-in-part of application No. 15/799,684, filed on Oct. 31, 2017.
(60) Provisional application No. 62/416,475, filed on Nov. 2, 2016.

(51) Int. Cl. A61K 31/4709; A61P 35/02; C12Q 1/6886

(52) U.S. Cl. CPC A61K 31/4709; A61P 35/02; C12Q 2600/156; C12Q 2600/118; C12Q 2600/106; C12Q 1/6886

(57) ABSTRACT

The present invention includes methods for treating a FLT3 mutated proliferative disorder comprising: measuring expression of a mutated FLT3 and a loss of function mutation in DNMT3A in a sample obtained from a tumor sample obtained from the patient, wherein the presence of the one or more genetic abnormalities indicates that the patient has a poor prognosis; and administering to the patient a therapeutically effective amount of crenolanib or a pharmaceutically acceptable salt thereof, wherein the crenolanib increases a chance of survival of the patient having both the mutated FLT3 and the loss of function mutation in DNMT3A, wherein the crenolanib, as shown below, is administered to a subject suffering from said disorder:

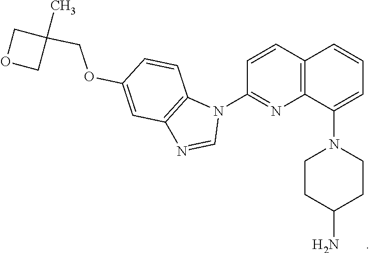 [SMILES: CC1(COc2ccc3c(c2)ncn3-c2ccc3cccc(N4CCC(N)CC4)c3n2)COC1]

### CROSS-REFERENCE TO RELATED APPLICATIONS

This application is a continuation-in-part of U.S. patent application Ser. No. 15/799,684 filed on Oct. 31, 2017, which claims priority to U.S. Provisional Application Ser. No. 62/416,475, filed Nov. 2, 2016, the entire contents of which are incorporated herein by reference.

### STATEMENT OF FEDERALLY FUNDED RESEARCH

Not applicable.

### TECHNICAL FIELD OF THE INVENTION

The present invention relates in general to the use of crenolanib in a pharmaceutically acceptable salt form for the treatment of proliferative disorder(s), characterized by mutations to particular tyrosine kinase pathways, and to a method of treatment of warm-blooded animals, preferably humans, in which a therapeutically effective dose of crenolanib is administered to a subject suffering from said proliferative disorder.

### INCORPORATION-BY-REFERENCE OF MATERIALS FILED ON COMPACT DISC

Not applicable.

### BACKGROUND OF THE INVENTION

Without limiting the scope of the invention, its background is described in connection with protein kinases.

Protein kinases are enzymes that chemically modify other proteins by catalyzing the transfer of gamma phosphates from nucleotide triphosphates, often adenosine triphosphate (ATP), and covalently attaching them to a free hydroxyl group of amino acid residues serine, threonine and tyrosine.

Approximately 30% of all human proteins may be modified by kinase activity. Protein kinases direct the enzymatic activity, cellular location and primary function/association of substrate proteins and regulate cell signal transduction and cell function coordination.

Research studies have shown that aberrant expression of normal or mutated protein kinases are frequently associated with the formation and propagation of a number of diseases. Studies have shown that overexpression or inappropriate protein kinase expression is associated with cancer, cardiovascular disease, rheumatoid arthritis, diabetes, ocular disease, neurologic disorders and autoimmune disease. Thus, investigating compounds that potently inhibit the activity and function of protein kinases will allow for a greater understanding of the physiological roles of protein kinases.

The FMS-like tyrosine kinase 3 (FLT3) gene encodes a membrane bound receptor tyrosine kinase that affects hematopoiesis leading to hematological disorders and malignancies. See Drexler, H G et al. Expression of FLT3 receptor and response to FLT3 ligand by leukemic cells. Leukemia. 1996; 10:588-599; Gilliland, D G and J D Griffin. The roles of FLT3 in hematopoiesis and leukemia. Blood. 2002;100:1532-1542; Stirewalt, D L and J P Radich. The role of FLT3 in hematopoietic malignancies. Nat Rev Cancer. 2003; 3:650-665. Activation of FLT3 receptor tyrosine kinases is initiated through the binding of the FLT3 ligand (FLT3L) to the FLT3 receptor, also known as Stem cell tyrosine kinase-1(STK-1) and fetal liver kinase-2 (flk-2), which is expressed on hematopoietic progenitor and stem cells.

FLT3 is one of the most frequently mutated genes in hematological malignancies, present in approximately 30% of adult acute myeloid leukemias (AML). See Nakao M, S Yokota and T Iwai. Internal tandem duplication of the FLT3 gene found in acute myeloid leukemia. Leukemia. 1996; 10:1911-1918; H Kiyoi, M Towatari and S Yokota. Internal Tandem duplication of the FLT3 gene is a novel modality of elongation mutation, which causes constitutive activation of the product. Leukemia.1998; 12:1333-1337; P D Kottaridis, R E Gale, et al. The presence of a FLT3 internal tandem duplication in patients with acute myeloid leukemia (AML) adds important prognostic information to cytogenetic risk group and response to the first cycle of chemotherapy: analysis of 854 patients from the United Kingdom Medical Research Council AML 10 and 12 trials. Blood. 2001; 98:1742-1759; Yamamoto Y, Kiyoi H, Nakano Y. Activating mutation of D835 within the activation loop of FLT3 in human hematologic malignancies. Blood. 2001;97:2434-2439; Thiede C, C Steudel, Mohr B. Analysis of FLT3-activating mutations in 979 patients with acute myelogenous leukemia: association with FAB subtypes and identification of subgroups with poor prognosis. Blood. 2002; 99:4326-4335. FLT3 mutations have been detected in approximately 2% of patients diagnosed with intermediate and high risk myelodysplastic syndrome (MDS). See S Bains, Luthra R, Medeiros L J and Zuo Z. FLT3 and NPM1 mutations in myelodysplastic syndromes:

Frequency and potential value for predicting progression to acute myeloid leukemia. American Journal of Clinical Pathology. January 2011; 135:62-69;P K Bhamidipati, Daver N G, Kantarjian H, et al. FLT3 mutations in myelodysplastic syndromes(MDS) and chronic myelomonocytic leukemia (CMML). 2012. Journal of Clinical Oncology. Suppl; abstract 6597. Like MDS, the number of FLT3 mutations in patients with acute promyelocytic leukemia (APL) is small. The most common FLT3 mutations are internal tandem duplications (ITDs) that lead to in-frame insertions within the juxtamembrane domain of the FLT3 receptor. FLT3-ITD mutations have been reported in 15-35% of adult AML patients. See Nakao M, S Yokota and T Iwai. Internal tandem duplication of the FLT3 gene found in acute myeloid leukemia. Leukemia. 1996; 10:1911-1918; H Kiyoi, M Towatari and S Yokota. Internal Tandem duplication of the FLT3 gene is a novel modality of elongation mutation, which causes constitutive activation of the product. Leukemia.1998;12:1333-1337; H Kiyoi, T Naoe and S Yokota. Internal tandem duplication of FLT3 associated with leukocytosis in acute promyelocytic leukemia. Leukemia Study Group of the Ministry of Health and Welfare (Kohseisho). Leukemia. 1997; 11:1447-1452; S Schnittger, C Schoch and M Duga. Analysis of FLT3 length mutations in 1003 patients with acute myeloid leukemia: correlation to cytogenetics, FAB subtype, and prognosis in the AMLCG study and usefulness as a marker for the detection of minimal residual disease. Blood. 2002;100:59-66. A FLT3-ITD mutation is an independent predictor of poor patient prognosis and is associated with increased relapse risk after standard chemotherapy, and decreased disease free and overall survival. See F M Abu-Duhier, Goodeve A C, Wilson G A, et al. FLT3 internal tandem duplication mutations in adult acute myeloid leukemia define a high risk group. British Journal of Haematology. 2000; 111:190-195; H Kiyoi, T Naoe, Y Nakano, et al. Prognostic implication of FLT3 and N-RAS gene mutations in acute myeloid leukemia. Blood. 1999; 93:3074-3080. Less frequent are FLT3 point mutations that arise in the activation loop of the FLT3 receptor. The most commonly affected codon is aspartate 835 (D835). Nucleotide substitutions of the D835 residue occur in approximately 5-10% of adult acute myeloid leukemia patients. See D L Stirewalt and J P Radich. The role of FLT3 in haematopoietic malignancies. Nature Reviews Cancer. 2003; 3:650-665; Y Yamamoto, H Kiyoi and Y Nakano, et al. Activating mutation of D835 within the activation loop of FLT3 in human hematologic malignancies. Blood. 2001; 97:2434-2439; C Thiede, Steudal C, Mohr B, et al. Analysis of FLT3-activating mutations in 979 patients with acute myelogenous leukemia: association with FAB subtypes and identification of subgroups with poor prognosis. Blood. 2002; 99:4326-4335; U Bacher, Haferlach C, W Kern, et al. Prognostic relevance of FLT3-TKD mutations in AML: the combination matters-an analysis of 3082 patients. Blood. 2008; 111:2527-2537.

The heightened frequency of constitutively activated mutant FLT3 in adult AML has made the FLT3 gene a highly attractive drug target in this tumor type. Several FLT3 inhibitors with varying degrees of potency and selectivity for the target have been or are currently being investigated and examined in AML patients. See T Kindler, Lipka D B, and Fischer T. FLT3 as a therapeutic target in AML: still challenging after all these years. Blood. 2010; 116:5089-102.

FLT3 kinase inhibitors known in the art include Lestaurtinib (also known as CEP 701, formerly KT-555, Kyowa Hakko, licensed to Cephalon); CHIR-258 (Chiron Corp.); EB10 and IMC-EB10 (ImClone Systems Inc.); Midostaurin (also known as PKC412, Novartis AG); Tandutinib (also known as MLN-518, formerly CT53518, COR Therapeutics Inc., licensed to Millennium Pharmaceuticals Inc.); Sunitinib (also known as SU11248, Pfizer USA); Quizartinib (also known as AC220, Ambit Biosciences); XL 999 (Exelixis USA, licensed to Symphony Evolution, Inc.); GTP 14564 (Merck Biosciences UK); AG1295 and AG1296; CEP-5214 and CEP-7055 (Cephalon). The following PCT International Applications and U.S. patent applications disclose additional kinase modulators, including modulators of FLT3: WO 2002032861, WO 2002092599, WO 2003035009, WO 2003024931, WO 2003037347, WO 2003057690, WO 2003099771, WO 2004005281, WO 2004016597, WO 2004018419, WO 2004039782, WO 2004043389, WO 2004046120, WO 2004058749, WO 2004058749, WO 2003024969 and U.S Patent Application Publication No. 2004/0049032. See also Levis M, K F Tse, et al. 2001 “A FLT3 tyrosine kinase inhibitor is selectively cytotoxic to acute myeloid leukemia blasts harboring FLT3 internal tandem duplication mutations.” Blood 98(3): 885-887; Tse K F, et al., Inhibition of FLT3-mediated transformation by use of a tyrosine kinase inhibitor. Leukemia. July 2001; 15 (7): 1001-1010; Smith, B. Douglas et al., Single agent CEP-701, a novel FLT3 inhibitor, shows biologic and clinical activity in patients with relapsed or refractory acute myeloid leukemia Blood, May 2004; 103: 3669-3676; Griswold, Ian J. et al., Effects of MLN518, A Dual FLT3 and KIT Inhibitor, on Normal and Malignant Hematopoiesis. Blood, November 2004; 104 (9): 2912-2918 [Epub ahead of print July 8]; Yee, Kevin W. H. et al., SU5416 and SU5614 inhibit kinase activity of wild-type and mutant FLT3 receptor tyrosine kinase. Blood, October 2002; 100(8): 2941-2949; O'Farrell, Anne-Marie et al., SU11248 is a novel FLT3 tyrosine kinase inhibitor with potent activity in vitro and in vivo. Blood, May 2003; 101(9): 3597-3605; Stone, R. M et al., PKC-412 FLT3 inhibitor therapy in AML: results of a phase II trials. Ann. Hematol. 2004; 83 Suppl 1:S89-90; and Murata, K. et al., Selective cytotoxic mechanism of GTP-14564, a novel tyrosine kinase inhibitor in leukemia cells expressing a constitutively active Fms-like tyrosine kinase 3 (FLT3). J Biol Chem. Aug. 29, 2003; 278 (35): 32892-32898 [Epub 2003 Jun. 18]; Levis, Mark et al., Small Molecule FLT3 Tyrosine Kinase Inhibitors. Current Pharmaceutical Design, 2004, 10, 1183-1193.

The aforementioned inhibitors have either been or are currently being investigated in the preclinical setting, or phase I and II trials as monotherapy in relapsed AML, or in phase III combination studies in relapsed AML. Despite reports of successful inhibition of FLT3 with these compounds in preclinical studies, complete remissions have rarely been achieved in FLT3 mutant AML patients in the clinical setting. For the majority of patients, the clinical response is short-lived. Response criteria for AML clinical trials are adapted from the International Working Group for AML. See Cheson et al. Revised Recommendations of the International Working Group for Diagnosis, Standardization of Response Criteria, Treatment Outcomes, and Reporting Standards for Therapeutic Trials in Acute Myeloid Leukemia. J Clin Oncol. 2003; 21: 4642-4649. Responders are patients who obtain a Complete Response (CR), Complete Response with incomplete blood count recovery (CRi), or Partial Remission (PR). Briefly, criteria are as follows:

1. Complete Remission (CR):
a. Peripheral blood counts:
   i. No circulating blasts
   ii. Neutrophil count≥1.0×10⁹/L
   iii. Platelet count≥100×10⁹/L
b. Bone marrow aspirate and biopsy:
   i. ≤5% blasts
   ii. No Auer Rods
   iii. No extramedullary leukemia

2. Complete remission with incomplete blood count recovery (CRi):
a. Peripheral blood counts:
   i. No circulating blasts
   ii. Neutrophil count<1.0×10⁹/L, or
   iii. Platelet count<100×10⁹/L
b. Bone marrow aspirate and biopsy
   i. ≤5% blasts
   ii. No Auer Rods
   iii. No extramedullary leukemia
3. Partial remission:
   a. All CR criteria if abnormal before treatment except:
   b. ≥50% reduction in bone marrow blast but still >5%

To date, clinical responses to FLT3 inhibitors have been primarily limited to clearance of peripheral blood (PB) blasts, which frequently return within a matter of weeks, while bone marrow (BM) blasts remain largely unaffected. For example, treatment with sorafenib, the prior mentioned multi-kinase inhibitor with activity against mutant FLT3, while effective in clearing PB blasts, has resulted in only modest BM blast reductions. See G Borthakur et al. Phase I study of sorafenib in patients with refractory or relapsed acute leukemias. Haematologica. January 2011; 96: 62-8. Epub 2010 Oct. 15. BM blast percentage plays a central role in the diagnosis and classification of AML. The presence of a heightened percentage of blasts in BM is associated with significantly shorter overall survival. See Small D. FLT3 mutations: biology and treatment. Hematology Am Soc Hematol Educ Program. 2006: 178-84; HM Amin et al. Having a higher blast percentage in circulation than bone marrow: clinical implications in myelodysplastic syndrome and acute lymphoid and myeloid leukemias. Leukemia. 2005; 19: 1567-72. To effectively treat FLT3 mutated AML patients and overcome the significant unmet need in this patient population, an inhibitor is required that significantly depletes both PB and BM blasts, bridges high risk and heavily pretreated patients to stem cell transplant, and can help to decrease relapse rates and increase overall survival in early stage disease patients.

Independent of the patient's FLT3 status, genetic abnormalities—including recurrent mutations, chromosomal aneuploidies and structural abnormalities—have historically played a critical role in characterizing the leukemia, helping determine disease aggressiveness, response to treatment, and prognosis. In the following table, “favorable risk” disease is associated with long-term survival of up to 65%, “intermediate risk” disease is associated with long-term survival of about 25%, and “adverse risk” disease is associated with long-term survival of less than 10%. See VanderWalde, A., “Genetics of Acute Myeloid Leukemia,” available at http://emedicine.medscape.com/article/1936033-overview (last updated 1 Apr. 2016).

| Risk Group | Genetic Abnormality |
|---|---|
| Favorable Risk | t(8; 21)(q22; q22.1); RUNX1-RUNX1T1 |
| | inv(16)(p13.1q22) or t(16; 16)(p13.1; q22); |
| | CBFB-MYH11 |
| | Mutated NPM1 without FLT3-ITD or with FLT3-ITD^{low} |
| | Biallelic mutated CEBPA |
| Intermediate | Mutated NPM1 and FLT3-ITD^{high} |
| Risk | Wild-type NPM1 without FLT3-ITD or with |
| | FLT3-ITD^{low} (without adverse-risk genetic lesions) |
| | t(9; 11)(p21.3; q23.3); MLLT3-KMT2A |
| | Cytogenetic abnormalities not classified as favorable |
| | or adverse |
| Adverse Risk | t(6; 9)(p23; q34.1); DEK-NUP214 |
| | t(v; 11q23.3); KMT2A rearranged |
| | t(9; 22)(q34.1; q11.2); BCR-ABL1 |
| | inv(3)(q21.3q26.2) or t(3; 3)(q21.3; q26.2); GATA2, |
| | MECOM(EVI1) |
| | −5 or del(5q); −7; −17/abn(17p) |
| | Complex karyotype, monosomal karyotype |
| | Wild-type NPM1 and FLT3-ITD^{high} |
| | Mutated RUNX1 |
| | Mutated ASXL1 |
| | Mutated TP53 |

See Döhner, H., et al. Diagnosis and management of AML in adults: 2017 ELN recommendations from an international expert panel. Blood. 2016; 129: 424-447.

Additionally, in the context of AML, clinicians and researchers have recently begun a progressive shift away from a morphologic classification scheme to one informed by causative genomic changes. See Papaemmanuil, E., et al. Genomic Classification and Prognosis in Acute Myeloid Leukemia. N Engl J Med. 2016; 374:2209-2221. Notably, a recent analysis of 1540 AML patients revealed 5234 “driver mutations” (using widely accepted genetic criteria for cancer-associated genes) involving 76 genes or regions within those patients, with mutation frequencies consistent with those found in previous studies. These driver mutations included recurrent fusion genes, aneuploidies, and leukemia gene mutations (such as base substitutions and small (200-bp) insertions or deletions), all found to display an effect on individual patient prognosis. At least 1 driver mutation was identified in 96% of patient samples, with 2 or more driver mutations found in 86% of patient samples. This comprehensive analysis led to the identification of previously unidentified leukemia-associated genes, as well as complex co-mutation patterns within these patient samples, indicating a renewed need to evaluate the prognoses of prospective AML patients in light of a renewed genomic classification scheme. Eleven genomic subgroups were thus proposed in light of this comprehensive study.

Overall survival in these patient samples was correlated with the number of driver mutations, independent of age and cell count. Through a multivariate model designed to explore the relative contributions of genetic, clinical, and diagnostic variables to overall survival, genomic features were determined to be the most powerful predictor of overall patient survival.

This study thus demonstrated considerable differences in clinical presentation and overall survival among the identified genomic subgroups. This finding, together with the discovery that the prognostic effects of individual mutations were significantly altered by the presence or absence of other driving mutations, suggests the necessity of assessing a number of driving mutations present in AML patients to provide a more comprehensive individual patient prognosis.

Consequently, the presence or absence of other driver lesions, including gene mutations, chromosomal aneuplodies, fusion genes, and complex karyotypes, has been demonstrated to provide a more comprehensive analysis of patient prognosis than the patient's status in one driver mutation alone. This is particularly true to the combination of activating FLT3 mutations and loss of function DNMT3A mutations. In light of this background, the need for the development of therapies capable of overcoming these particularly grim patient prognoses takes on a renewed importance.

The current invention seeks to overcome disadvantages of the prior art.

### SUMMARY OF THE INVENTION

In one embodiment, the present invention includes a method for treating a human patient having leukemia with crenolanib, wherein the human patient is suffering from FLT3 mutated leukemia, the method comprising: determining whether the human patient has a poor prognosis for the FLT3 mutated leukemia by: obtaining or having obtained a leukemia sample from the human patient; and performing or having performed a genotyping assay on the biological sample to determine if the human patient has both a mutated FLT3 or a constitutively active FLT3 mutant and one or more loss of function mutations in DNMT3A, wherein the presence of both the mutated FLT3 and the one or more loss of function mutations in DNMT3A indicates that the patient has a poor prognosis; and if the human patient has the poor prognosis administering to the patient a therapeutically effective amount of crenolanib or a pharmaceutically acceptable salt thereof, to treat the leukemia. In one aspect, the FLT3 mutation is selected from at least one of FLT3-ITD or FLT3-TKD. In another aspect, the one or more loss of function mutation in the DNMT3A gene is selected from a nonsense mutation or frameshift mutation at or before amino acid residue R885, or missense mutations at amino acid residues between M666 and R885 inclusive in the catalytic domain or combinations thereof. In another aspect, the therapeutically effective amount of crenolanib or the pharmaceutically acceptable salt thereof are from about 50 to 500 mg per day, 100 to 450 mg per day, 200 to 400 mg per day, 300 to 500 mg per day, 350 to 500 mg per day, or 400 to 500 mg per day; or the therapeutically effective amount of crenolanib or the pharmaceutically acceptable salt thereof is administered at least one of continuously, intermittently, systemically, or locally; or the therapeutically effective amount of crenolanib or the pharmaceutically acceptable salt thereof is administered orally, intravenously, or intraperitoneally. In another aspect, the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, and crenolanib succinate. In another aspect, the therapeutically effective amount of crenolanib or the pharmaceutically acceptable salt thereof is: administered up to three times or more a day for as long as the human patient is in need of treatment for the leukemia; or provided at least one of sequentially or concomitantly, with another pharmaceutical agent in a newly diagnosed human leukemia patient, to maintain remission of an existing human leukemia patient, or in a relapsed/refractory human leukemia patient; or provided as a single agent or in combination with another pharmaceutical agent in a patient with a newly diagnosed leukemia, to maintain remission, or in a relapse/refractory human leukemia patient; or provided as a single agent or in combination with another pharmaceutical agent in a newly diagnosed human pediatric leukemia patient, to maintain remission, or in a relapsed/refractory human pediatric leukemia patient. In another aspect, the human patient is relapsed/refractory to another tyrosine kinase inhibitor or chemotherapy.

In another embodiment, the present invention includes a method for treating a human patient suffering from a leukemia comprising: identifying that the human patient is in need of therapy for the leukemia, wherein the leukemia comprises both a deregulated FLT3 receptor tyrosine kinase and a loss of function mutation in DNMT3A, and wherein the leukemia is characterized by having a poor prognosis; and administering to the patient a therapeutically effective amount of crenolanib or a salt thereof sufficient to treat the leukemia. In one aspect, the FLT3 mutation is selected from at least one of FLT3-ITD or FLT3-TKD. In another aspect, the loss of function mutation in the DNMT3A gene is selected from a nonsense mutation or frameshift mutation at or before amino acid residue R885, or missense mutations at amino acid residues between M666 and R885 inclusive in the catalytic domain or combinations thereof. In another aspect, the therapeutically effective amount of crenolanib or the pharmaceutically acceptable salt thereof is administered orally, intravenously, or intraperitoneally. In another aspect, the therapeutically effective amount of crenolanib or the pharmaceutically acceptable salt thereof is: at least one of crenolanib besylate, crenolanib phosphate, crenolanib lactate, crenolanib hydrochloride, crenolanib citrate, crenolanib acetate, crenolanib toluenesulphonate, and crenolanib succinate; or is provided at least one of sequentially or concomitantly, with a chemotherapeutic agent in a newly diagnosed leukemia, to maintain remission, or in a relapsed/refractory leukemia; or is provided as a single agent or in combination with a chemotherapeutic agent for treatment of a human pediatric patient with leukemia; or is provided at least one of sequentially or concomitantly to at least one of post standard induction therapy, or high dose induction therapy, in newly diagnosed leukemia; or is provided as a single agent in treatment of patients with the leukemia that is either refractory to, or has relapsed after, prior treatment with a chemotherapeutic agent. In another aspect, the patient is refractory to at least one other tyrosine kinase inhibitor or a chemotherapy.

In another embodiment, the present invention includes a method for treating a human patient suffering from acute myelogenous leukemia (AML) comprising: obtaining a sample from the human patient; determining from the sample that the human patient has AML with a deregulated FLT3 receptor or a constitutively active FLT3 receptor; determining if the AML has one or more loss of function mutations in the DNMT3A gene; and if the AML has both the deregulated FLT3 receptor or a constitutively active FLT3 receptor and the loss of function mutation in the DNMT3A gene, then administering to the human patient in need of such treatment a therapeutically effective amount of crenolanib or salt thereof. In one aspect, the FLT3 mutation is selected from at least one of FLT3-ITD or FLT3-TKD. In another aspect, the loss of function mutation in the DNMT3A gene is selected from a nonsense mutation or frameshift mutation at or before amino acid residue R885, or missense mutations at amino acid residues between M666 and R885 inclusive in the catalytic domain or combinations thereof.

In another embodiment, the present invention includes a method for specifically inhibiting a deregulated or constitutively active FLT3 receptor tyrosine kinase, comprising: obtaining a sample from a human patient having acute myelogenous leukemia (AML); determining that the AML has a FLT3 receptor tyrosine kinase that is deregulate or constitutively active and one or more loss of function mutations in the DNMT3A gene; wherein the AML with both the deregulated or constitutively active FLT3 receptor tyrosine kinase and the one or more loss of function mutations in the DNMT3A gene cause a poor prognosis; and if the human patient has AML with both the deregulate or constitutively active FLT3 receptor tyrosine kinase and the one or more loss of function mutations in the DNMT3A gene, administering to the human patient in need of such treatment a therapeutically effective amount of crenolanib or a salt thereof, sufficient to eliminate the AML. In one aspect, the deregulated FLT3 receptor is selected from at least one of FLT3-ITD or FLT3-TKD. In another aspect, the one or more loss of function mutation in the DNMT3A gene is selected from a nonsense mutation or frameshift mutation at or before amino acid residue R885, or missense mutations at amino acid residues between M666 and R885 inclusive in the catalytic domain or combinations thereof. In another aspect, the therapeutically effective amount of crenolanib or the salt thereof is provided: in an amount that decreased the human patient's circulating peripheral blood blast count; or in an amount that decreases the human patient's bone marrow blast count; or in an amount from about 50 to 500 mg per day, 100 to 450 mg per day, 200 to 400 mg per day, 300 to 500 mg per day, 350 to 500 mg per day, or 400 to 500 mg per day; or in an amount that is delivered at least one of continuously, intermittently, systemically, or locally. In another aspect, the therapeutically effective amount of crenolanib or the salt thereof is administered orally, intravenously, or intraperitoneally. In another aspect, the crenolanib or the salt thereof is at least one of crenolanib besylate, crenolanib phosphate, crenolanib lactate, crenolanib hydrochloride, crenolanib citrate, crenolanib acetate, crenolanib toluenesulphonate, and crenolanib succinate. In another aspect, the therapeutically effective amount of crenolanib or the salt thereof is at least one of: administered up to three times or more a day for as long as the human patient is in need of treatment; or is provided at least one of sequentially or concomitantly, with another pharmaceutical agent in a newly diagnosed AML, to maintain remission, or in a relapsed/refractory AML; or the crenolanib or the salt thereof is provided as a single agent or in combination with another pharmaceutical agent in a newly diagnosed AML, to maintain remission, or in a relapsed/refractor AML; or the therapeutically effective amount of crenolanib or the salt thereof is provided as a single agent or in combination with another pharmaceutical agent in a newly diagnosed AML pediatric patient, to maintain remission, or in a relapsed/refractory AML pediatric patient. In another aspect, the patient is relapsed/refractory to a prior tyrosine kinase inhibitor.

This summary of the invention does not necessarily describe all necessary features of the invention.

### BRIEF DESCRIPTION OF THE DRAWINGS

Not applicable.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention comprises the use of the compounds of the present invention to treat disorders related to FLT3 kinase activity or expression in a subject.

Crenolanib (4-Piperidinamine, 1-[2-[5-[(3-methyl-3-oxetanyl) methoxy]-1H-benzimidazol-1-yl]-8-quinolinyl]) and its pharmaceutically acceptable salts, are protein tyrosine kinase inhibitors selective for constitutively active FLT3 mutations, including FLT3 ITD and FLT3 TKD mutations. Unlike prior FLT3 inhibitors in the art, the besylate salt form of crenolanib has been shown to be remarkably effective in depleting circulating peripheral blood blast percentages and bone marrow blast percentages in heavily pretreated FLT3 mutant AML patients without significantly increasing patient QT prolongation. Crenolanib is currently being investigated for use in the treatment of patients with relapsed or refractory constitutively activated FLT3 mutated primary AML or AML secondary to myelodysplastic syndrome.

An analysis of Crenolanib's efficacy in patients presenting with concomitant FLT3 mutations, as well as other cytogenetic or molecular abnormalities, are also presently being developed through ongoing clinical trials.

Crenolanib safety and tolerability was evaluated between November 2003 and September 2006 in a phase I first-in-human dose-escalation single agent study in heavily pretreated patients with advanced solid tumors (Protocol A5301001; See N Lewis et al., J Clin Oncol. 2009; 27: p5262-5269). Fifty-nine patients were enrolled and completed the study. Most treatment related adverse events were of grade 1 or 2 severity. There was no evidence of cumulative toxicity. In patients treated with lower drug dosages ranging from 60-200 mg once daily, the most common adverse events observed were grade 1 nausea and vomiting, which usually occurred approximately 45 minutes after dosing. There were no grade 3 or 4 toxicities in these patients. At higher doses 280 mg and 340 mg once daily, liver enzyme elevations were the most severe side effects. Liver enzyme levels returned to normal following the discontinuation of crenolanib. The present invention has demonstrated that the administration of 100 mg three times daily of crenolanib besylate to human patients diagnosed with constitutively activated FLT3 mutant relapsed or refractory AML does not always result in an elevation of liver enzymes. See Example two in Examples section of this patient application. It also demonstrates that when liver enzymes are elevated that liver enzyme levels can be decreased by discontinuing the drug for approximately 1 week and re-starting crenolanib at a reducing dosage of 80 mg three times daily.

No grade 2/3/4 QT prolongation was observed in any of the 59 patients treated in the phase I dose escalation safety study, despite crenolanib dose received. Similarly, there have been no significant differences in baseline QT prolongation and on-treatment QT prolongation in a currently ongoing pediatric glioma trial with twenty-four children being treated with the besylate form of crenolanib. Likewise, the present invention has shown no cases of QT prolongation following the administration of 100 mg of crenolanib besylate three times daily to human patients diagnosed with constitutively activated FLT3 mutant relapsed or refractory AML. Other FLT3 inhibitors known in the art have caused significant QTc prolongation leading to strict clinical study inclusion and exclusion criteria to prevent severe adverse events. For example, two separate quizartinib AML studies have revealed that the compound causes significant Q prolongation. In a 76 patient phase I single agent study evaluating the compound in both FLT3 wildtype and FLT ITD mutated relapsed and refractory AML identified QT prolongation as the dose limiting toxicity. See J Cortes et al. AC220, a potent, selective, second generation FLT3 receptor tyrosine kinase (RTK) inhibitor, in a first-in-human (FIH) phase I AML study. Blood (ASH Annual Meeting Abstracts) 2009 November. Additionally, interim data from a phase II trial of quizartinib monotherapy in 62 patients with relapsed or refractory AML with FLT3 ITD activating mutations asymptomatic QT prolongation was one of the most common (>19%) drug related adverse events. QT prolongation of all grades occurred in 21 (34%) patients. More than half of the QT prolongation events recorded were grade 3 (18%). Reducing the starting dose of quizartinib by greater than 30% did not alleviate all cases of QT prolongation. See J Cortes et al. A phase II open-label, AC220 monotherapy efficacy study in patients with refractory/relapsed FLT3-ITD positive acute myeloid leukemia: updated interim results. Blood (ASH Annual Meeting Abstracts) 2011 December.

The present invention includes methods of treating proliferative disorders in a subject. In one embodiment, the present invention includes a method for treating a human patient having leukemia with crenolanib, wherein the human patient is suffering from FLT3 mutated leukemia, the method comprising: determining whether the human patient has a poor prognosis for the FLT3 mutated leukemia by: obtaining or having obtained a leukemia sample from the human patient; and performing or having performed a genotyping assay on the biological sample to determine if the human patient has both a mutated FLT3 or a constitutively active FLT3 mutant and one or more loss of function mutations in DNMT3A, wherein the presence of both the mutated FLT3 and the one or more loss of function mutations in DNMT3A indicates that the patient has a poor prognosis; and if the human patient has the poor prognosis administering to the patient a therapeutically effective amount of crenolanib or a pharmaceutically acceptable salt thereof, to treat the leukemia. In another embodiment, the present invention includes a method for treating a human patient suffering from a leukemia comprising: identifying that the human patient is in need of therapy for the leukemia, wherein the leukemia comprises both a deregulated FLT3 receptor tyrosine kinase and a loss of function mutation in DNMT3A, and wherein the leukemia is characterized by having a poor prognosis; and administering to the patient a therapeutically effective amount of crenolanib or a salt thereof sufficient to treat the leukemia. In another embodiment, the present invention includes a method for treating a human patient suffering from acute myelogenous leukemia (AML) comprising: obtaining a sample from the human patient; determining from the sample that the human patient has AML with a deregulated FLT3 receptor or a constitutively active FLT3 receptor; determining if the AML has one or more loss of function mutations in the DNMT3A gene; and if the AML has both the deregulated FLT3 receptor or a constitutively active FLT3 receptor and the loss of function mutation in the DNMT3A gene, then administering to the human patient in need of such treatment a therapeutically effective amount of crenolanib or salt thereof. In another embodiment, the present invention includes a method for specifically inhibiting a deregulated or constitutively active FLT3 receptor tyrosine kinase, comprising: obtaining a sample from a human patient having acute myelogenous leukemia (AML); determining that the AML has a FLT3 receptor tyrosine kinase that is deregulate or constitutively active and one or more loss of function mutations in the DNMT3A gene; wherein the AML with both the deregulated or constitutively active FLT3 receptor tyrosine kinase and the one or more loss of function mutations in the DNMT3A gene cause a poor prognosis; and if the human patient has AML with both the deregulate or constitutively active FLT3 receptor tyrosine kinase and the one or more loss of function mutations in the DNMT3A gene, administering to the human patient in need of such treatment a therapeutically effective amount of crenolanib or a salt thereof, sufficient to eliminate the AML.

The present inventor recognized that multiple studies have demonstrated that patients with both FLT3 and DNMT3A mutations have worse outcomes than patients with either mutation alone. See Ley, T.J., “DNMT3A Mutations in Acute Myeloid Leukemia,” New England J. Med. Vol. 363, No. 25, pp. 2424-2433 (16 Dec. 2010). See Papaemmanuil, E., et al. Genomic Classification and Prognosis in Acute Myeloid Leukemia. N Engl J Med. 2016; 374:2209-2221. See Patel, J.P et al. Prognostic Relevance of Integrated Genetic Profiling in Acute Myeloid Leukemia. N Engl J Med. Vol 366, No. 12, pp 1079-1089 (22 Mar. 2012). In contrast to FLT3, in which activating mutations are oncogenic, it is loss of function mutations in DNMT3A which are associated with disease. DNMT3A, or DNA (ctyosine-5)-methyltransferase 3A, is an epigentic regulator which methylates CpG islands within the regulatory regions of DNA. DNMT3A functions as a homotetramer. The tetramerization interface involves interaction of amino acide resiudes 666-885 in two DNMT3A molecules, with tight ring stacking. See Holz-Schietinger, C. et al. Mutations in DNA Methyltransferase (DNMT3A) Observed in Acute Myeloid Luekemia Patients Disrupt Processive Methylation. J Biol Chem. Vol. 287, No. 37, pp. 30941-51 (23 Jun. 2012). Missense mutations at these residues disrupt normal tetramerization, resulting in significantly decreased processivity and methyltransferase activity.

Missense mutations in the tetramerization interface represent the most common DNMT3A mutations found in AML. However, there are a number of other mutation types that also result in loss of function. Specifically, nonsense (stop-gain) and frameshift mutations. Table 1 below contains an analysis of the DNMT3A mutations found by

Papaemmanuil et al. Over 85% of the mutations identified in DNMT3A are loss of function mutations that result in haploinsufficiency.

DNMT3A Mutations by Type Identified by Papaemmanuil

| | | Percentage of |
|---|---|---|
| | | DNMT3A |
| Mutation Type | n | Mutations |
| Nonsense/Stop-gain | 11 | 2.9% |
| Frameshift | 27 | 7.2% |
| Oligomerization interface | 288 | 76.6% |
| (666-885) | | |

The functional consequencies of DNMT3A haploinsufficiency have been extensively studied in preclinical models. Loss of DNMT3A results in hypomethylation of target genes, and aberrant expression of proteins. In animal models, 56% of mice with DNMT3A haploinsufficiency develop hematological malignancies such as AML within 2 years. See Cole, C. B. et al. Haploinsuffciency for DNA Methyltransferase 3A Predisposes Hematopoietic Cells to Myeloid Malignances. J Clin. Invest. Vol. 127, No. 10, pp. 3657-3674 (6 Sept 2017). When DNMT3A haploinsufficiency is combined with FLT3-ITD, it results in a fully penetrant, highly aggressive AML phenotype. See Meyer, S. E. et al. DNMT3A Haploinsufficiency Transforms FLT3-ITD Myeloproliferative Disease into a Rapid, Spontaneous, and Fully Penetrant Acute Myeloid Leukemia. Canceer Discov. Vol. 6, No. 5, pp. 501-515 (27 Mar 2016).

As used herein, the term “poor prognosis” refers to a decreased chance of survival (for example, decreased overall survival, relapse-free survival, or metastasis-free survival). For example, a poor prognosis has a decreased chance of survival includes a survival time of equal to or less than 60 months, such as 50 months, 40 months, 30 months, 20 months, 12 months, 6 months, or 3 months from time of diagnosis or first treatment or remission.

By contrast, a “good prognosis” refers to an increased chance of survival, for example increased overall survival, relapse-free survival, or metastasis-free survival. For example, a good prognosis has an increased chance of survival includes a survival time of at least 60 months from time of diagnosis, such as 60 months, 80 months, 100 months, 120 months, 150 months, or more from time of diagnosis or first treatment.

Detection of the mutated FLT3 and/or one or more genetic abnormalities can be performed using any suitable means known in the art. For example, detection of gene mutations can be accomplished by detecting nucleic acid molecules (such as DNA) using nucleic acid amplification methods (such as RT-PCR) or high-throughput sequencing (i.e. “next-generation sequencing”). Detection of chromosomal abnormalities can also be accomplished using karyotyping or in situ hybridization that detect structural and numerical alterations.

In mutated FLT3 tumors, the alteration in expression or presence of one or more genetic abnormalities, such as, e.g., chromosomal translocations, deletions, alternative gene splicing, mutations or deletions within coding or intron-exon boundary regions, can be lead to a measurable decrease in prognosis. In addition to a pre-existing FLT3 mutation, the additional genetic abnormalities disclosed herein significantly decrease the prognosis of the patient. A poor prognosis can refer to any negative clinical outcome, such as, but not limited to, a decrease in likelihood of survival (such as overall survival, relapse-free survival, or metastasis-free survival), a decrease in the time of survival (e.g., less than 5 years, or less than one year), presence of a malignant tumor, an increase in the severity of disease, a decrease in response to therapy, an increase in tumor recurrence, an increase in metastasis, or the like. In particular examples, a poor prognosis is a decreased chance of survival (for example, a survival time of equal to or less than 60 months, such as 50 months, 40 months, 30 months, 20 months, 12 months, 6 months or 3 months from time of diagnosis or first treatment).

In other embodiments of the method, the presence of the one or more loss of function mutations in DNMT3A (in addition to the FLT3 mutation) in the tumor sample relative to a control indicates a poor prognosis for the patient with the tumor. The method includes detecting the presence of one or more loss of function mutations in DNMT3A, using genetic sequencing techniques known in the art to determine if a sample has one of the specified DNMT3A mutations (nonsense, frameshift, or missense within the oligomerization interface).

As used herein, the phrases “mutations responsible for cancer” and “driver mutations” are used interchangeably to refer to mutations that are present in cancer tissues and which are capable of inducing carcinogenesis of cells. Generally, if a mutation is found in a cancer tissue in which no other known oncogene mutations exists (in other words, if a mutation exists in a mutually exclusive manner with known oncogene mutations), then the mutation can be determined to be a responsible mutation for cancer, and thus, a “driver mutation”. Driver mutations within DNMT3A include loss of function mutations resulting from nonsense, frameshift, or missense mutations within the oligomerization interface.

In one embodiment, the present invention provides a method for reducing or inhibiting the kinase activity of FLT3 in a subject comprising the step of administering a compound of the present invention to the subject.

As used herein, the term “subject” or “patient” are used interchangeable to refer to an animal, such as a mammal or a human, who has been the object of medical treatment, observation or experiment.

In one embodiment to this aspect, the present invention provides a method for reducing or inhibiting the kinase activity of FLT3 in a subject comprising the step of administering a compound of the present invention to the subject.

The term “subject” refers to an animal, such as a mammal or a human, who has been the object of treatment, observation or experiment.

In other embodiments to this aspect, the present invention provides therapeutic methods for treating a subject with a cell proliferative disorder driven by aberrant kinase activity of mutant FLT3. In one example, the invention provides methods for treating a cell proliferative disorder related to mutant FLT3, comprising administration of a therapeutically effective amount of a pharmaceutical composition comprising a compound of the present invention in a subject. Administration of said therapeutic agent can occur upon manifestation of symptoms characteristic of the FLT3 driven cell proliferative disorder, such that a disease or disorder treated.

The term “therapeutically effective amount” as used herein, refers to an amount of active compound or pharmaceutical salt that elicits the biological or medicinal response in a subject that is being sought by a researcher, veterinarian, medical doctor or other clinician, which includes alleviation of the symptoms of the disease or disorder being treated.

Methods for determining therapeutically effective doses for pharmaceutical compositions comprising a compound of the present invention are known in the art.

As used herein, the term “composition” is intended to encompass a product comprising the specified ingredients in the specified amounts, as well as any product which results, directly or indirectly, from combinations of the specified ingredients in the specified amounts.

As used herein, the terms “disorder related to FLT3,” or “disorders related to FLT3 receptor,” or “disorders related to FLT3 receptor tyrosine kinase,” or “FLT3 driven cell proliferative disorder” includes diseases associated with or implicating FLT3 activity, for example, mutations leading to constitutive activation of FLT3. Examples of “disorders related to FLT3” include disorders resulting from over stimulation of FLT3 due to mutations in FLT3, or disorders resulting from abnormally high amount of FLT3 activity due to abnormally high amount of mutations in FLT3. It is known that over-activity of FLT3 has been implicated in the pathogenesis of many diseases, including the following listed cell proliferative disorders, neoplastic disorders and cancers.

The term “cell proliferative disorders” refers to excess cell proliferation of one or more subset of cells in a multicellular organism resulting in harm (i.e. discomfort or decreased life expectancy) to the multicellular organism. Cell proliferative disorders can occur in different types of animals and humans. As used herein, “cell proliferative disorders” include neoplastic disorders.

The term “neoplastic disorder” as used herein, refers to a tumor resulting from abnormal or uncontrolled cellular growth. Examples of neoplastic disorders include, but are not limited to the following disorders, for instance: the myeloproliferative disorders, such as thrombocytopenia, essential thrombocytosis (ET), agnogenic myeloid metaplasia, myelofibrosis (MF), myelofibrosis with myeloid metaplasia (MMM), chronic idiopathic myelofibrosis (UIMF), and polycythemia vera (PV), the cytopenias, and pre-malignant myelodysplastic syndromes; cancers such as glioma cancers, lung cancers, breast cancers, colorectal cancers, prostate cancers, gastric cancers, esophageal cancers, colon cancers, pancreatic cancers, ovarian cancers, and hematological malignancies, including myelodysplasia, multiple myeloma, leukemias, and lymphomas. Examples of hematological malignancies include, for instance, leukemias, lymphomas, Hodgkin's disease, and myeloma. Also, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocytic leukemia (JMML), adult T-cell ALL, AML, with trilineage myelodysplasia (AMLITMDS), mixed lineage leukemia (MLL), myelodysplastic syndromes (MDSs), myeloproliferative disorders (MPD), and multiple myeloma (MM).

The expression of mutated FLT3, constitutively active FLT3 mutant, and the one or more mutations in DNMT3A, can be determined using standard molecular biology techniques, including sequencing at the RNA or DNA level, protein expression, protein function, the presence or absence of the RNA, DNA, and/or protein, as will be known to those of skill in the art following the teachings of, e.g., standard techniques for sequencing (including Next Generation Sequencing (NGS)), cloning, RNA and DNA isolation, amplification and purification, detection and identification of chromosomal abnormalities, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; Fitchen, et al. (1993) Annu Rev. Microbiol. 47:739-764; Tolstoshev, et al. (1993) in Genomic Research in Molecular Medicine and Virology, Academic Press; and Ausubel et al. (1992) Current Protocols in Molecular Biology, Greene/Wiley, New York, N.Y. Abbreviations and nomenclature, where employed, are deemed standard in the field and commonly used in professional journals such as those cited herein. The above techniques can be used to detect genetic abnormalities such as aneuploidy, monosomy, trisomy, or polysomy; chromosomal aberrations such as one or more deletions, duplications, translocations, inversions, insertions, rings, or isochromosomes. Additional genetic abnormalities include driver mutations such as those selected from at least one loss of function in DNMT3A, human or animal, with the names available from Genecards.com, with current accession numbers, sequences, and probes to the same incorporated herein by reference.

For example, detection of gene mutations can be accomplished by detecting nucleic acid molecules (such as DNA) using nucleic acid amplification methods (such as RT-PCR) or high throughput sequencing (i.e., “next-generation sequencing”). By example, next-generation sequencing platforms such as Illumina may be used to determine the exact genetic sequence of specific genes, or portions of genes, of interest. In brief, DNA from a tumor sample is fragmented, ligated with the appropriate primers and adaptors, and amplified using PCR during “library preparation”. The prepared libraries are then sequenced using one of a number of commercially available systems which generates the sequence of the chosen target genes, all exomes, or the entire genome. The sequences are then analyzed using commercially available software, which aligns the tumor sample sequence to the known sequence of the genes of interest and performs a variant calling step, which identifies differences at the DNA level in the tumor sample and determines if such mutations would result in alteration of the amino acid sequence in the translated protein. Using these systems, a person of skill in the art can determine if a subject has one of the identified mutations with in FLT3. Further information on FLT3, including full gene and protein sequences, known clinically relevant variants and mutations, tissue expression, and signaling interaction partners can be found at UniProt (accession number P36888-1), GenBank (accession number NM 04119.2), and GenPept (accession number NP 004110.2).

The types of driver mutations in the DNMT3A gene include loss of function mutations which lead to haploinsufficiency. The specific mutations include nonsense or frameshift mutations before amino acid residue R885, and missense mutations within the oligomerization interface (amino acid residues 666-885, inclusive). The presence of one or more of these mutations can be determined using standard molecular biology techniques, including Next Generation Sequencing. Standard analysis methods, including variant calling and such analyses as SIFT (Sorting Intolerant from Tolerant) and PolyPhen (Polymorphism Phenotyping), may be used to determine if a particular variant found in a tumor sample is detrimental to normal protein function. Gene mutation panels such as those used by medical professionals during diagnostic workup of potential proliferative disease patients may also be used. Further information on DNMT3A, including full gene and protein reference sequences and known clinically relevant variants and mutations can be found at UniProt (accession number Q9Y6K1-1), Gen Bank (accession number NM_{—} 022552.4), and GenPept (accession number NP_072046.2).

In a further embodiment, the present invention can be combined with another therapy as a combination therapy for treating or inhibiting the onset of a cell proliferative disorder related to FLT3 in a subject. The combination therapy comprises the administration of a therapeutically effective amount of a compound of the present invention and one or more other anti-cell proliferation therapies including, but not limited to, chemotherapy and radiation therapy.

In an embodiment of the present invention, a compound of the present invention may be administered in combination with chemotherapy. Used herein, chemotherapy refers to a therapy involving a chemotherapeutic agent. A variety of chemotherapeutic agents may be used in combination with the present invention. By way of example only, taxane compounds, specifically docetaxel, is safely administered in combination with a compound of the present invention in a dosage of 75 mg per square meter (mg/m²) of body surface area.

Chemotherapy is known to those skilled in the art. The appropriate dosage and scheme for chemotherapy will be similar to those already employed in clinical therapies wherein the chemotherapy is delivered in combination with other therapies or used alone.

In another embodiment of the present invention, compounds of the present invention may be administered in combination with radiation therapy. Used herein, “radiation therapy” refers to a therapy that comprises the exposure of a subject in need to radiation. Radiation therapy is known to those skilled in the art. The appropriate dosage and scheme for radiation therapy will be similar to those already employed in clinical therapies wherein the radiation therapy is delivered in combination with other therapies or used alone.

In another embodiment of the present invention, the compounds of the present invention may be administered in combination with a targeted therapy. As used herein, “targeted therapy” refers to a therapy targeting a particular class of proteins involved in tumor development or oncogenic signaling. For example, tyrosine kinase inhibitors against vascular endothelial growth factor have been used in treating cancers.

The present invention also includes methods that include the use of a second pharmaceutical agent in addition to compounds of the present invention, the two may be administered simultaneously or sequentially (in either order).

In one embodiment, the present invention therapeutically effective amounts of the compound having formula I:

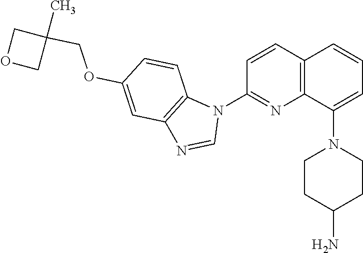 [SMILES: CC1(COc2ccc3c(c2)ncn3-c2ccc3cccc(N4CCC(N)CC4)c3n2)COC1]

or a pharmaceutically acceptable salt or solvate thereof, in a therapeutically or prophylactically effective amount against a proliferative disease is selected from at least one of a leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy. Pharmaceutically acceptable salts including hydrochloride, phosphate and lactate are prepared in a manner similar to the benzenesulfonate salt and are well known to those of moderate skill in the art.

Compounds of the present invention may be administered to a subject systemically, for example, orally, intravenously, subcutaneously, intramuscular, intradermal or parenterally. The compounds of the present invention can also be administered to a subject locally.

Compounds of the present invention may be formulated for slow-release or fast-release with the objective of maintaining contact of compounds of the present invention with targeted tissues for a desired range of time.

Compositions suitable for oral administration include solid forms, such as pills, tablets, caplets, capsules, granules, and powders, liquid forms, such as solutions, emulsions, and suspensions. Forms useful for parenteral administration include sterile solutions, emulsions and suspensions.

The daily dosage of the compounds of the present invention may be varied over a wide range from 50 to 500 mg per adult human per day. For oral administration, the compositions are preferably provided in the form of tablets containing 20 and 100 milligrams. The compounds of the present invention may be administered on a regimen up to three times or more per day. Preferably three times per day. Optimal doses to be administered may be determined by those skilled in the art, and will vary with the compound of the present invention used, the mode of administration, the time of administration, the strength of the preparation, the details of the disease condition. Factors associated with patient characteristics, such as age, weight, and diet will call for dosage adjustments.

Preparation of the compounds of the present invention. General synthetic methods which may be referred to for preparing the compounds of formula I are provided in U.S. Pat. No. 5,990,146 (issued Nov. 23, 1999) (Warner-Lambert Co.) and PCT published application numbers WO 99/16755 (published Apr. 8, 1999) (Merck & Co.) WO 01/40217 (published Jul. 7, 2001) (Pfizer, Inc.), US Patent Application No. US 2005/0124599 (Pfizer, Inc.) and U.S. Pat. No. 7,183,414 (Pfizer, Inc.), relevant portions incorporated herein by reference.

Pharmaceutically acceptable salts such as hydrochloride, phosphate and lactate are prepared in a manner similar to the benzenesulfonate salt and are well known to those of moderate skill in the art. The following representative compounds of the present invention are for exemplary purposes only and are in no way meant to limit the invention, including Crenolanib as Crenolanib Besylate, Crenolanib Phosphate, Crenolanib Lactate, Crenolanib Hydrochloride, Crenolanib Citrate, Crenolanib Acetate, Crenolanib Toluenesulphonate and Crenolanib Succinate.

### SUMMARY OF EXAMPLES

### Example A

The leukemic blasts from a newly diagnosed patient harbored in addition to FLT3-ITD and TKD mutations, a mutation in NPM1, and a loss of function missense mutation in DNMT3A. The patient achieved reduction in bone marrow blasts to less than 5% following induction combination chemotherapy followed by sequential administration of crenolanib besylate.

### Example B

The leukemic blasts from a newly diagnosed patient harbored in addition to a FLT3-ITD mutation, a loss of function frameshift mutation in the DNMT3A gene. The patient achieved reduction in bone marrow blasts to less than 5% following induction combination chemotherapy followed by sequential administration of crenolanib besylate.

### Example C

The leukemic blasts from a newly diagnosed patient harbored in addition to a FLT3-ITD mutation, a loss of function missense mutation in the DNMT3A gene. The patient achieved reduction in bone marrow blasts to less than 5% following induction combination chemotherapy followed by sequential administration of crenolanib besylate.

### Example D

The leukemic blasts from a relapsed/refractory patient harbored in addition to a FLT3-ITD mutation, a loss of function frameshift mutation in the DNMT3A gene. The patient achieved reduction in bone marrow blasts to less than 5% following salvage combination chemotherapy followed by sequential administration of crenolanib besylate.

### Example E

The leukemic blasts from a relapsed/refractor patient with prior TKI failure harbored in addition to FLT3-ITD and TKD mutations, a loss of function point mutation in the DNMT3A gene. The patient achieved reduction in bone marrow blasts to less than 5% following crenolanib besylate monotherapy.

### Example F

The leukemic blasts from a newly diagnosed patient harbored in addition to a FLT3-ITD mutation, a loss of function frameshift mutation in the DNMT3A gene. The patient achieved reduction in bone marrow blasts to less than 5% following induction combination chemotherapy followed by sequential administration of crenolanib besylate and was bridged to an allogeneic hematopoietic stem cell transplantation (HSCT).

### Example G

The leukemic blasts from a relapsed/refractory patient w harbored, in addition to a FLT3-ITD mutation, a loss of function missense mutation in the DNMT3A gene. The patient achieved reduction in bone marrow blasts to less than 5% following crenolanib besylate monotherapy

### Example H

The leukemic blasts from a relapsed/refractory patient with prior TKI failure harbored in addition to FLT3-ITD and TKD mutations, mutations in the NPM1, and WT1 genes, and a loss of function point mutation in DNMT3A gene and an abnormal complex karyotype. The patient achieved reduction in bone marrow blasts to less than 5% following crenolanib besylate monotherapy and was bridged to an allogeneic hematopoietic stem cell transplantation (HSCT).

### Example A

Effect of Crenolanib Besylate Therapy in a Newly Diagnosed AML Patient with FLT3-ITD, FLT3-TKD, NPM1, and DNMT3A Mutations with Normal Karyotype: Achievement of reduction in bone marrow blasts to less than 5% with hematologic recovery.

A 54-year-old female was diagnosed with AML positive for both FLT3-ITD and FLT3-TKD mutations. The patient's leukemic blasts also had mutations in the NPM1 and DNMT3A genes. Specifically, the patient's leukemic blasts had a R882C mutation in DNMT3A, a missense mutation within the oligomerization interface and the most common DNMT3A mutation found in AML. As the FLT3-ITD, NPM1, and DNMT3A mutations are characterized as independent driver mutations, and are together associated with a particularly poor prognosis, the patient's presentation of these triple mutations placed her in a significantly high-risk group for AML patients, associated with poor response rates, increased cumulative incidence of relapse, and shortened survival. Half of patients with these mutations are expected to die within 1 year of diagnosis. See Papaemmanuil, E., “Genomic Classification and Prognosis in Acute Myeloid Leukemia,” New England J. Med. Vol. 374, No. 23, pp. 2209-2221 (9 June 2016).

At diagnosis, the patient was found to have 63% bone marrow blasts. Following diagnosis, the patient was provided with oral crenolanib besylate on a clinical trial newly diagnosed AML patients (NCT02283177). The patient was initially treated with induction chemotherapy, comprised of seven days of cytarabine and three days of daunorubicin; the patient began therapy with 100 mg of crenolanib besylate three times daily on day 10. A bone marrow biopsy taken on day 35 of the clinical trial revealed the patient's bone marrow blasts had reduced to less than 5%, classified as a complete remission. The patient remains alive and free of disease more than four years after start of therapy.

Table A below illustrates the ability of crenolanib to clear and maintain clearance of malignant leukemia in the bone marrow of Example A, a newly diagnosed AML patient with FLT3-ITD, NPM1, and DNMT3A mutations with normal karyotype after treatment with chemotherapy and crenolanib besylate.

TABLE A
Effect of crenolanib to clear malignant leukemia in Example A.

| Days on Clinical Trial | Bone Marrow Blast (%) |
|---|---|
| 0 | 63% |
| 35 | <5% |
| 238 | <5% |
| 294 | <5% |
| 406 | <5% |

### Example B

Effect of Crenolanib Besylate Therapy in a Newly Diagnosed AML Patient with FLT3-ITD and DNMT3A Mutations: Achievement of reduction in bone marrow blasts to less than 5% with incomplete hematologic recovery.

A 57-year-old female was diagnosed with AML positive for FLT3-ITD and DNMT3A mutations. Specifically, the patient had a frameshift mutation at F290 in the DNMT3A gene. This combination of mutations placed her in a high risk group for AML patients, associated with poor response rates, increased cumulative incidence of relapse, and shortened survival. See Ley, T. J., “DNMT3A Mutations in Acute Myeloid Leukemia,” New England J. Med. Vol. 363, No. 25, pp. 2424-2433 (16 Dec. 2010).

At diagnosis, the patient was found to have 55% bone marrow blasts. Following diagnosis, the patient was provided with oral crenolanib besylate on a clinical trial newly diagnosed AML patients (NCT02283177). The patient was initially treated with induction chemotherapy, comprised of seven days of cytarabine and three days of idarubicin; the patient began therapy with 100 mg of crenolanib besylate three times daily on day 9.

A bone marrow biopsy taken on day 33 of the clinical trial revealed the patient's bone marrow blasts had reduced to less than 5%, classified as a complete remission with incomplete hematological recovery. The patient died shortly after achieving remission due to underlying liver disease.

Table B below illustrates the ability of crenolanib to clear malignant leukemia in the bone marrow of Example B, a newly diagnosed AML patient with FLT3-ITD and DNMT3A mutations after treatment with chemotherapy and crenolanib besylate.

TABLE B
Effect of crenolanib to clear malignant leukemia in Example B.

| Days on Clinical Trial | Bone Marrow Blast (%) |
|---|---|
| 0 | 55% |
| 33 | <5% |

### Example C

Effect of Crenolanib Besylate Therapy in a Newly Diagnosed AML Patient with FLT3-ITD and DNMT3A Mutations: Achievement of reduction in bone marrow blasts to less than 5% with hematologic recovery.

A 47-year-old male was diagnosed with AML positive for FLT3-ITD and DNMT3A mutations. Specifically, the patient had a S714C mutation in DNMT3A, which is within the oligomerization interface and would result in loss of function and haploinsufficiency. These mutations independently categorize her as a high-risk AML patient, which is associated with poor response rate, increased cumulative incidence of relapse, and shortened survival. The patient's presentation of these combined mutational characteristics placed him in a higher risk group than the FLT3-ITD mutation alone. See Ley, T. J., “DNMT3A Mutations in Acute Myeloid Leukemia,” New England J. Med. Vol. 363, No. 25, pp. 2424-2433 (16 Dec. 2010).

At diagnosis, the patient was found to have 63% bone marrow blasts. Following diagnosis, the patient was provided with oral crenolanib besylate on a clinical trial newly diagnosed AML patients (NCT02283177). The patient was initially treated with induction chemotherapy, comprised of seven days of cytarabine and three days of idarubicin; the patient began therapy with 100 mg of crenolanib besylate three times daily on day 9.

A bone marrow biopsy taken on day 38 of the clinical trial revealed the patient's bone marrow blasts had reduced to less than 5%, classified as a complete remission, and the presence of FLT3-ITD mutation was no longer detectable. After completing induction chemotherapy, the patient received one cycle of chemotherapy consolidation, followed by 6 months of maintenance crenolanib besylate monotherapy. The patient remains alive and free of disease for more than 4 years after start of therapy. Less than 20% of these patients would be expected to survive greater than 2 years after diagnosis. See Ley, T. J., “DNMT3A Mutations in Acute Myeloid Leukemia,” New England J. Med. Vol. 363, No. 25, pp. 2424-2433 (16 Dec. 2010).

Table C below illustrates the ability of crenolanib to clear and maintain clearance of malignant leukemia in the bone marrow of Example C, a newly diagnosed AML patient with FLT3-ITD and DNMT3A mutations after treatment with chemotherapy and crenolanib besylate.

TABLE C
Effect of crenolanib on malignant leukemia in Example C.

| Days on Clinical Trial | Bone Marrow Blast (%) |
|---|---|
| 0 | 63% |
| 38 | <5% |

### Example D

Effect of Crenolanib Besylate Therapy in a Relapsed/Refractory AML Patient with a FLT3-ITD Mutation and DNMT3A mutation: Achievement of reduction in bone marrow blasts to less than 5% with hematologic recovery.

A 75-year-old male was diagnosed with AML positive for a FLT3-ITD mutation and harbored DNMT3A mutation. Specifically, the patient harbored a frameshift mutation at F827 in the DNMT3A gene, which would result in loss of function and haploinsufficiency. These mutations independently categorize him as a high-risk AML patient, which is associated with poor response rate, increased cumulative incidence of relapse, and shortened survival. The patient's presentation of these combined mutational characteristics placed him in a higher risk group than the FLT3-ITD mutation alone. See Ley, T. J., “DNMT3A Mutations in Acute Myeloid Leukemia,” New England J. Med. Vol. 363, No. 25, pp. 2424-2433 (16 Dec. 2010).

Following his diagnosis, the patient was initially treated with induction chemotherapy. Approximately seven months later the patient had relapsed with a bone marrow blast percentage of 39%.

Further genetic analysis showed that the patient had acquired a trisomy 8 chromosomal abnormality in addition to the FLT3-ITD and (6;9) translocation that were present upon initial diagnosis. As trisomy 8 has been also been characterized as an independent driver mutation separate and apart from other driver mutations characteristic of AML (such as the patient's FLT3 mutational status and (6; 9) translocation) and has itself been associated with a poor prognosis, the patient's presentation of these three characteristics placed him in a significantly higher risk group.

At the time of relapse, the patient was provided with oral crenolanib besylate on a clinical trial for relapsed or refractory AML patients (NCT02626338). The patient was initially treated with salvage chemotherapy, comprised of six days of cytarabine and three days of mitoxantrone; the patient began therapy with 100 mg of crenolanib besylate three times daily on day 8.

A bone marrow biopsy taken on day 34 of the clinical trial revealed the patient's bone marrow blasts had reduced to less than 5%, classified as a complete remission. Further analysis revealed that the patient now exhibited a normal male karyotype. The patient remained in remission for more than 400 days. Less than 10% of relapsed patients with these mutations treated with standard therapy would be expected to survive for more than 400 days. See Levis, M., et al. Results from a randomized trial of salvage chemotherapy followed by lestaurtinib for patients with FLT3 mutant AML in first relapse. Blood. 2011; 117:3294-3301.

Table D below illustrates the ability of crenolanib to clear and maintain clearance malignant leukemia in the bone marrow of Example D, a relapsed/refractory AML patient with a FLT3-ITD mutation, DNMT3A mutation, trisomy 8, and (6;9) translocation after treatment with salvage chemotherapy and crenolanib besylate.

TABLE D
Effect of crenolanib on malignant leukemia in Example D.

| Days on Clinical Trial | Bone Marrow Blast (%) |
|---|---|
| 0 | 39% |
| 34 | <5% |
| 115 | <5% |

No additional bone marrow biopsies were performed on study after day 115, as the patient remained in remission.

### Example E

Effect of Crenolanib Besylate Therapy in a Relapsed/Refractory AML Patient with FLT3-ITD, FLT3-TKD, and DNMT3A Mutations: Achievement of reduction in bone marrow blasts to less than 5% with incomplete hematologic recovery.

A 60-year-old male was diagnosed with AML positive for FLT3-ITD and DNMT3A mutations and initially treated with induction chemotherapy and a TKI. The patient was refractory to chemotherapy and subsequently received sorafenib, a FLT3 inhibitor, due the presence of a FLT3-ITD mutation. The patient initially responded to sorafenib, however relapsed after less than 2 months. The patient's history or multiple relapses, prior TKI therapy, and FLT3-ITD, FLT3-TKD, and DNMT3A mutations this patient in a high-risk group.

At the time of relapse following sorafenib therapy, the patient was provided with oral crenolanib besylate on a clinical trial for relapsed or refractory AML patients (NCT01657682). The patient began therapy at 100 mg TID of crenolanib besylate monotherapy.

At the time of study entry, the patient had a FLT3-ITD mutation and a R882H mutation in the DNMT3A gene, a missense mutation in the oligomerization interface that disrupts tetramerization and results in loss of function and haploinsufficiency. This combination of mutations places this patient in a high-risk group. See Ley, T. J., “DNMT3A Mutations in Acute Myeloid Leukemia,” New England J. Med. Vol. 363, No. 25, pp. 2424-2433 (16 Dec. 2010).

Table E below illustrates the ability of crenolanib to clear malignant leukemia in the bone marrow of Example E, a relapsed/refractory AML patient with FLT3-ITD and DNMT3A mutations who failed prior FLT3 TKI after treatment with crenolanib besylate monotherapy.

TABLE E
Effect of crenolanib on malignant leukemia in Example E.

| Days on Clinical Trial | Bone Marrow Blast (%) |
|---|---|
| 0 | 17% |
| 27 | <5% |

### Example F

Effect of Crenolanib Besylate Therapy in a Newly Diagnosed AML Patient with FLT3-ITD and DNMT3A Mutations: Achievement of reduction in bone marrow blasts to less than 5% with hematologic recovery.

A 23-year-old female was diagnosed with AML with FLT3-ITD and DNMT3A mutations. Specifically, the patient had a frameshift mutation at N802, which would result in loss of function and haploinsufficiency. This combination of mutations places this patient in a high-risk group. See Ley, T. J., “DNMT3A Mutations in Acute Myeloid Leukemia,” New England J. Med. Vol. 363, No. 25, pp. 2424-2433 (16 Dec. 2010).

At diagnosis, the patient was found to have 70% bone marrow blasts. Following diagnosis, the patient was provided with oral crenolanib besylate on a clinical trial for newly diagnosed AML patients (NCT02283177). The patient was initially treated with induction chemotherapy, comprised of seven days of cytarabine and three days of daunorubicin; the patient began therapy with 100 mg of crenolanib besylate three times daily on day 9.

A bone marrow biopsy taken on day 36 of the clinical trial revealed the patient's bone marrow blasts had reduced to less than 5%, classified as a complete remission.

The patient remains alive and free of disease more than 4 years after start of therapy. Less than 20% of patients with FLT3 and DNMT3A mutations treated with standard therapy would be expected to survive without disease for more than 2 years. See Ley, T. J., “DNMT3A Mutations in Acute Myeloid Leukemia,” New England J. Med. Vol. 363, No. 25, pp. 2424-2433 (16 Dec. 2010).

Table F below illustrates the ability of crenolanib to clear malignant leukemia in the bone marrow of Example F, a newly diagnosed AML patient with FLT3-ITD and DNTM3A mutations.

TABLE F
Effect of crenolanib on malignant leukemia in Example F.

| Days on Clinical Trial | Bone Marrow Blast (%) |
|---|---|
| 0 | 70% |
| 36 | <5% |
| 97 | <5% |
| 121 | <5% |

### Example G

Effect of Crenolanib Besylate Therapy in a Relapsed/Refractory AML Patient with a FLT3-TKD Mutation and DNMT3A mutation: Achievement of reduction in bone marrow blasts to less than 5% with hematologic recovery.

A 51-year-old female was diagnosed with AML positive for a FLT3-TKD mutation (D835). The patient was initially treated with induction chemotherapy and achieved remission. However, she relapsed 10 months later. At relapse, the patient was found to have gained a FLT3-ITD mutation, and also maintained a DNMT3A mutation. Specifically, the patient harbored a R882H mutation in DNMT3A, a missense mutation within the oligomerization interface which results in loss of function and haploinsufficiency.

At the time of relapse, the patient was provided with oral crenolanib besylate on a clinical trial for relapsed or refractory AML patients (NCT01657682). The patient began therapy at 100 mg TID of crenolanib besylate monotherapy.

A bone marrow biopsy taken on day 28 of the clinical trial revealed the patient's bone marrow blasts had reduced to less than 5%, classified as a complete remission. The patient was then bridged to an allogenic hematopoietic stem cell transplant.

Table G below illustrates the ability of crenolanib to clear malignant leukemia in the bone marrow of Example G, a relapsed/refractory AML patient with FLT3-ITD mutation and DNMT3A mutation after treatment with salvage chemotherapy and crenolanib besylate.

TABLE G
Effect of crenolanib on malignant leukemia in Example G.

| Days on Clinical Trial | Bone Marrow Blast (%) |
|---|---|
| 0 | 76% |
| 28 | <5% |

### Example H

Effect of Crenolanib Besylate Therapy in a Relapsed/Refractory AML Patient with FLT3-ITD and TKD mutations and DNMT3A: Achievement of reduction in bone marrow blasts to less than 10% with hematologic recovery.

A 48-year-old female was diagnosed with AML positive for a FLT3-ITD mutation and normal female karyotype and initially treated with induction chemotherapy. Approximately seven months later, the patient relapsed and received multiple salvage therapies including chemotherapy and the tyrosine kinase inhibitor, sorafanib.

The patient did not respond to these therapies and had 46% bone marrow blasts following treatment, which blasts were positive for FLT3-ITD, FLT3-TKD, NPM1, DNMT3A, and WT1 mutations. Specifically, the patient had a R882H mutation in DNMT3A, a missense mutation within the oligomerization interface which would result in loss of function and haploinsufficiency. Presence of concurrent FLT3-ITD, NPM1, and DNMT3A mutations and a WT1 mutation both placed the patient in a high-risk group. The patient was further shown to exhibit an abnormal complex karyotype characterized by translocations (1;6), (4; 19), and (10; 15). As complex karyotype has been characterized as an independent driver mutation separate and apart from other driver mutations characteristic of AML (such as the patient's FLT3 mutational status) and has itself been associated with a poor prognosis, the patient's presentation of these three sets of characteristics placed her in a significantly higher risk group.

The patient was then provided with oral crenolanib besylate as part of a clinical trial of single-agent crenolanib in relapsed or refractory AML patients (NCT01657682). The patient began therapy with 200 mg/m² per day of crenolanib besylate.

A bone marrow biopsy taken on day 29 of the clinical trial revealed the patient's bone marrow blasts had reduced to 7%, classified as a partial remission.

Table H below illustrates the ability of crenolanib to significantly reduce malignant leukemia in the bone marrow of Example H, a relapsed/refractory AML patient with FLT3-ITD and WT1 mutations and complex karyotype with prior TKI failure.

TABLE H
Effect of crenolanib on malignant leukemia in Example H.

| Days on Clinical Trial | Bone Marrow Blast (%) |
|---|---|
| 0 | 46% |
| 29 | 7% |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains.

All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

For each of the claims, each dependent claim can depend both from the independent claim and from each of the prior dependent claims for each and every claim so long as the prior claim provides a proper antecedent basis for a claim term or element.

### REFERENCES

DREXLER, et al. “Expression of FLT3 receptor and response to FLT3 ligand by leukemic cells” Leukemia. Apr. 10, 1996; 10:588-599 (ABSTRACT ONLY).

GILLILAND, et al. “The roles of FLT3 in hematopoiesis and leukemia.” Blood. Sep. 1, 2002; 100: 1532-1542.

STIREWALT, et al. “The role of FLT3 in haematopoietic malignancies” Nature Reviews Cancer. 2003; 3:650-665.

NAKAO, et al. ABSTRACT ONLY: “Internal tandem duplication of the FLT3 gene found in acute myeloid leukemia.” Leukemia. 1996; 10:1911-1918.

KIYOI, et al. “Internal tandem duplication of the FLT3 gene is a novel modality of elongation mutation which causes constitutive activation of the product” Leukemia. 1998; 12:1333-1337.

KOTTARIDIS, et al. “The presence of a FLT3 internal tandem duplication in patients with acute myeloid leukemia (AML) adds important prognostic information to cytogenetic risk group and response to the first cycle of chemotherapy: analysis of 854 patients from the United Kingdom Medical Research Counsel AML 10 and 12 trials” Blood. Sep. 15, 2001; 98: 1742-1759.

YAMAMOTO, et al. “Activating mutation of D835 within the activation loop of FLT3 in human hematologic malignancies” Blood. 2001; 97″2434-2439.

THIEDE, et al. “Analysis of FLT3-activating mutations in 979 patients with acute myelogenous leukemia: association with FAB subtypes and identification of subgroups with poor prognosis” Blood. 2002; 99:4326-4335.

BAINS, et al. “FLT3 and NPM1 mutations in myelodysplastic syndromes: Frequency and potential value for predicting progression to acute myeloid leukemia” American Journal of Clinical Pathology. January 2011; 135: 62-69.

BHAMIDPATI, et al. “FLT3 mutations in myelodysplastic syndromes (MDS) and chronic myelomonocytic leukemia” (CMML). 2012. Journal of Clinical Oncology. Suppl; abstract 6597.

KIYOI, et al. “Internal tandem duplication of FLT3 associated with leukocytosis in acute promyelocytic leukemia” Leukemia Study Group of the Ministry of Health and Welfare (Kohseisho). Leukemia. 1997; 11: 1447-1452.

SCHNITTGER, et al. “Analysis of FLT3 length mutations in 1003 patients with acute myeloid leukemia: correlation to cytogenetics, FAB subtype, and prognosis in the AMLCG study and usefulness as a marker for the detection of minimal residual disease” Blood. 2002; 100: 59-66.

ABU-DUHIER, et al. “FLT3 internal tandem duplication mutations in adult acute myeloid leukemia define a high-risk group” British Journal of Hematology. Jun.7, 2000; 111: 190-195.

KIYOI et al. “Prognostic implication of FLT3 and N-RAS gene mutations in acute myeloid leukemia” Blood. May 1, 1999; 93:3074-3080.

BACHER, et al. “Prognostic relevance of FLT3-TKD mutations in AML: the combination matters—an analysis of 3082 patients” Blood. Mar. 1, 2008; 111:2527-2537.

KINDLER, et al. “FLT3 as a therapeutic target in AML: still challenging after all these years” Blood. Dec. 9, 2010; 116:5089-102.

LEVIS, et al. “A FLT3 tyrosine kinase inhibitor is selectively cytotoxic to acute myeloid leukemia blasts harboring FLT3 internal tandem duplication mutations” Blood. Aug. 1, 2001; 98(3): 885-887.

SMITH, et al. Single agent CEP-701, a novel FLT3 inhibitor, shows biologic and clinical activity in patients with relapsed or refractory acute myeloid leukemia Blood, May 2004; 103: 3669-3676.

GRISWOLD, et al. “Effects of MLN518, a dual FLT3 and KIT inhibitor, on normal and malignant hematopoiesis” Blood. Nov. 2004; 104 (9): 2912-2918.

YEE, et al., SU5416 and SU5614 inhibit kinase activity of wild-type and mutant FLT3 receptor tyrosine kinase. Blood, Octpber 2002; 100(8): 2941-2949.

O'Farrell et al., SU11248 is a novel FLT3 tyrosine kinase inhibitor with potent activity in vitro and in vivo. Blood, May 2003; 101(9): 3597-3605.

Murata, K. et al., Selective cytotoxic mechanism of GTP-14564, a novel tyrosine kinase inhibitor in leukemia cells expressing a constitutively active Fms-like tyrosine kinase 3 (FLT3). J Biol Chem. Aug. 29, 2003; 278 (35): 32892-32898 [Epub 2003 Jun. 18].

Stone, R. M et al., PKC-412 FLT3 inhibitor therapy in AML: results of a phase II trials. Ann. Hematol. 2004; 83 Suppl 1:S89-90.

CHESON, et al. “Revised Recommendations of the International Working Group for Diagnosis, Standardization of Response Criteria, Treatment Outcomes, and Reporting Standards for Therapeutic Trials in Acute Myeloid Leukemia” J Clin Oncol. Dec. 15, 2003; 21: 4642-4649.

LEVIS, et al. “Small Moleucle FLT3 Tyrosine Kinase Inhibitors” Current Pharmaceutical Design. 2004, 10, 1183-1193.

BORTHAKUR, et al. “Phase I study of sorafenib in patients with refractory or relapsed acute leukemias.” Haematologica. Jan. 2011; 96: 62-8. Epub Oct. 15, 2010.

Small D. FLT3 mutations: biology and treatment. Hematology Am Soc Hematol Educ Program. 2006: 178-84.

AMIN, et al. “Having a higher blast percentage in circulation than bone marrow; clinical implications in myelodysplastic syndrome and acute lymphoid and myeloid leukemias” Leukemia. July 28, 2005: 19: 1567-72.

VANDERWALDE, A., “Genetics of Acute Myeloid Leukemia,” available at http://emedicine.medscape.com/article/1936033-overview (last updated 1 Apr. 2016).

PAPAEMMANUIL, E., “Genomic Classification and Prognosis in Acute Myeloid Leukemia,” New England J. Med. Vol. 374, No. 23, pp. 2209-2221 (9 Jun. 2016).

DÖHNER, H., et al. “Diagnosis and management of AML in adults: 2017 ELN recommendations from an international expert panel,” Blood. Vol. 129, No. 3, pp. 424-447 (28 Nov. 2016).

BAKSHI, S., et al. “Trisomy 8 in leukemia: A GCRI experience,” Indian J Hum Genet. 2012;18:106-108.

LEY, T. J., et al. “DNMT3A Mutations in Acute Myeloid Luekemia,” New England J. Med. Vol. 363, No. 25, pp. 2424-2433 (16 Dec. 2010).

PATEL, J. P., et al. “Prognostic Relevance of Integrated Genetic Profiling in Acute Myeloid Leukemia,” New England J. Med. Vol 366, No. 12, pp. 1079-189 (22 Mar. 2012).

HOLZ-SCHIETINGER, C. et al., “Mutations in DNA Methyltransferase (DNMT3A) Observed in Acute Myeloid Leukemia Patients Disrupt Processive Methylation,” J Biol Chem, Vol. 287, No. 37, pp. 30941-51 (23 Jun. 2012).

COLE, C. B. et al., “Haploinsufficiency for DNA Methyltransferase 3A Predisposes Hematopoietic Cells to Myeloid Malignancies,” J Clin Invest., Vol 127, No. 10, pp. 3657-74 (6 Sep. 2017).

MEYER, S. E. et al., “DNMT3A Haploinsufficiency Transforms FLT3-ITD Myeloproliferative Disease into a Rapid, Spontaneous, and Fully Penetrant Acute Myeloid Leukemia,” Cancer Discov, Vol. 6, No. 5, pp. 501-15 (27 Mar. 2016).

LEWIS, et al. “Phase I study of the safety, tolerability, and pharmacokinetics of oral CP-868-596, a highly specific platelet-derived growth factor receptor tyrosine kinase inhibitor in patients with advanced cancers” J Clin Oncol. Nov. 1, 2009; 27(31), 5262-5269.

CORTES, et al. “AC220, a potent, selective, second generation FLT3 receptor tyrosine kinase (RTK) inhibitor, in a first-in-human (FIH) phase I AML study” Blood (ASH Annual Meeting Abstracts) 2009 November.

CORTES, et al. “A phase II open-label, AC220 monotherapy efficacy study in patients with refractory/relapsed FLT3-ITD positive acute myeloid leukemia: updated interim results” Blood (ASH Annual Meeting Abstracts) 2011 December.

MENDLER, J., “RUNX1 Mutations Are Associated With Poor Outcome in Younger and Older Patients With Cytogenetically Normal Acute Myeloid Leukemia and With Distinct Gene and MicroRNA Expression Signatures,” J. Clin. Oncol. Vol. 30, No. 25, pp. 3109-3118 (2 Jul. 2012).

DICKER, F., et al. “Trisomy 13 is strongly associated with AML1/RUNX1 mutations and increased FLT3 expression in acute myeloid leukemia,” Blood. 2007; 110:1308-1316.

HEROLD, T., et al. “Isolated trisomy 13 defines a homogeneous AML subgroup with high frequency of mutations in spliceosome genes and poor prognosis,” Blood. 2014; 124:1304-1311.

LEVIS, M., et al. “Results from a randomized trial of salvage chemotherapy followed by lestaurtinib for patients with FLT3 mutant AML in first relapse,” Blood. 2011; 117:3294-3301.

PASCHKA, P., et al. “Wilms' Tumor 1 Gene Mutations Independently Predict Poor Outcome in Adults With Cytogenetically Normal Acute Myeloid Leukemia: A Cancer and Leukemia Group B Study,” J Clin Oncol. 2008; 26:4595-4602.

What is claimed is:

1. A method for treating a human patient having leukemia with crenolanib, wherein the human patient is suffering from a leukemia with a mutated or a constitutively active FLT3 and one or more loss of function mutations in a DNMT3A gene, the method comprising:
determining whether the human patient has a poor prognosis for the leukemia by:
obtaining or having obtained a biological sample from the human patient; and
performing or having performed a genotyping or phenotyping assay on the biological sample to determine if the human patient has both the mutated or constitutively active FLT3 and the one or more loss of function mutations in the DNMT3A gene, wherein a presence of both the mutated constitutively active FLT3 and the one or more loss of function mutations in the DNMT3A gene indicates that the human patient has a poor prognosis; and
if the human patient has the poor prognosis administering to the human patient a therapeutically effective amount of crenolanib or a pharmaceutically acceptable salt thereof, to treat the leukemia.

2. The method of claim 1, wherein the mutated or constitutively active FLT3 is selected from at least one of FLT3-ITD or FLT3-TKD.

3. The method of claim 1, wherein the one or more loss of function mutation in the DNMT3A gene is selected from a nonsense mutation or frameshift mutation at or before amino acid residue R885, or missense mutations at amino acid residues between M666 and R885 inclusive in a catalytic domain or combinations thereof.

4. The method of claim 1, wherein the therapeutically effective amount of crenolanib or the pharmaceutically acceptable salt thereof are from about 50 to 500 mg per day, 100 to 450 mg per day, 200 to 400 mg per day, 300 to 500 mg per day, 350 to 500 mg per day, or 400 to 500 mg per day; or
the therapeutically effective amount of crenolanib or the pharmaceutically acceptable salt thereof is administered at least one of continuously, intermittently, systemically, or locally; or
the therapeutically effective amount of crenolanib or the pharmaceutically acceptable salt thereof is administered orally, intravenously, or intraperitoneally.

5. The method of claim 1, wherein the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, and crenolanib succinate.

6. The method of claim 1, wherein the method further comprises at least one of:
administering up to three times or more a day for as long as the human patient is in need of treatment for the leukemia; or
providing at least one of sequentially or concomitantly, with another pharmaceutical agent in a newly diagnosed human leukemia patient, to maintain remission of an existing human leukemia patient, or in a relapsed/refractory human leukemia patient; or
providing as a single agent or in combination with another pharmaceutical agent in the human patient with a newly diagnosed leukemia, to maintain remission, or in a relapse/refractory human leukemia patient; or
providing as a single agent or in combination with another pharmaceutical agent in a newly diagnosed human pediatric leukemia patient, to maintain remission, or in a relapsed/refractory human pediatric leukemia patient.

7. The method of claim 1, wherein the human patient is relapsed/refractory to another tyrosine kinase inhibitor or chemotherapy.

8. A method for treating a human patient suffering from a leukemia comprising:
identifying that the human patient is in need of therapy for the leukemia, wherein the leukemia comprises both a deregulated FLT3 receptor tyrosine kinase and a loss of function mutation in a DNMT3A gene, and wherein the leukemia is characterized by having a poor prognosis; and
administering to the human patient a therapeutically effective amount of crenolanib or a salt thereof sufficient to treat the leukemia with the deregulated FLT3 receptor tyrosine kinase and loss of function mutation in the DNMT3A gene.

9. The method of claim 8, wherein the deregulated FLT3 receptor tyrosine kinase is selected from at least one of FLT3-ITD or FLT3-TKD.

10. The method of claim 8, wherein the loss of function mutation in the DNMT3A gene is selected from a nonsense mutation or frameshift mutation at or before amino acid residue R885, or missense mutations at amino acid residues between M666 and R885 inclusive in a catalytic domain or combinations thereof.

11. The method of claim 8, wherein the therapeutically effective amount of crenolanib or a pharmaceutically acceptable salt thereof is administered orally, intravenously, or intraperitoneally.

12. The method of claim 8, wherein the method further comprises at least one of:
providing the crenolanib as at least one of crenolanib besylate, crenolanib phosphate, crenolanib lactate, crenolanib hydrochloride, crenolanib citrate, crenolanib acetate, crenolanib toluenesulphonate, and crenolanib succinate; or
providing at least one of sequentially or concomitantly, with a chemotherapeutic agent in a newly diagnosed leukemia, to maintain remission, or in a relapsed/refractory leukemia; or
providing as a single agent or in combination with a chemotherapeutic agent for treatment of a human pediatric patient with leukemia; or
providing at least one of sequentially or concomitantly to at least one of post standard induction therapy, or high dose induction therapy, in newly diagnosed leukemia; or
providing as a single agent in treatment of human patients with the leukemia that is either refractory to, or has relapsed after, prior treatment with a chemotherapeutic agent.

13. The method of claim 8, wherein the human patient is refractory to at least one other tyrosine kinase inhibitor or a chemotherapy.

14. A method for treating a human patient suffering from acute myelogenous leukemia (AML) comprising:
obtaining a sample from the human patient;
determining from the sample that the human patient has AML with a deregulated FLT3 receptor or a constitutively active FLT3 receptor;
determining if the AML has one or more loss of function mutations in a DNMT3A gene; and
if the AML has both the deregulated FLT3 receptor or the constitutively active FLT3 receptor and the loss of function mutation in the DNMT3A gene, then administering to the human patient in need of such treatment a therapeutically effective amount of crenolanib or salt thereof.

15. The method of claim 14, wherein the deregulated FLT3 receptor or the constitutively active FLT3 receptor is selected from at least one of FLT3-ITD or FLT3-TKD.

16. The method of claim 14, wherein the loss of function mutation in the DNMT3A gene is selected from a nonsense mutation or frameshift mutation at or before amino acid residue R885, or missense mutations at amino acid residues between M666 and R885 inclusive in a catalytic domain or combinations thereof.

17. A method for specifically inhibiting a deregulated or constitutively active FLT3 receptor tyrosine kinase, comprising:
obtaining a sample from a human patient having acute myelogenous leukemia (AML);
determining that the AML has a FLT3 receptor tyrosine kinase that is deregulate or constitutively active and one or more loss of function mutations in a DNMT3A gene;
wherein the AML with both the deregulated or constitutively active FLT3 receptor tyrosine kinase and the one or more loss of function mutations in the DNMT3A gene cause a poor prognosis; and
if the human patient has AML with both the deregulated or constitutively active FLT3 receptor tyrosine kinase and the one or more loss of function mutations in the DNMT3A gene, administering to the human patient in need of such treatment a therapeutically effective amount of crenolanib or a salt thereof, sufficient to eliminate the AML.

18. The method of claim 17, wherein the deregulated or constitutively active FLT3 receptor tyrosine kinase is selected from at least one of FLT3-ITD or FLT3-TKD.

19. The method of claim 17, wherein the one or more loss of function mutation in the DNMT3A gene is selected from a nonsense mutation or frameshift mutation at or before amino acid residue R885, or missense mutations at amino acid residues between M666 and R885 inclusive in a catalytic domain or combinations thereof.

20. The method of claim 17, wherein the method further comprises at least one of:
providing crenolanib or the salt thereof in an amount that decreased a circulating peripheral blood blast count in the human patient; or
providing crenolanib or the salt thereof in an amount that decreases a bone marrow blast count in the human patient; or
providing crenolanib or the salt thereof in an amount from about 50 to 500 mg per day, 100 to 450 mg per day, 200 to 400 mg per day, 300 to 500 mg per day, 350 to 500 mg per day, or 400 to 500 mg per day; or
providing crenolanib or the salt thereof in an amount that is delivered at least one of continuously, intermittently, systemically, or locally.

21. The method of claim 17, wherein the therapeutically effective amount of crenolanib or the salt thereof is administered orally, intravenously, or intraperitoneally.

22. The method of claim 17, wherein the crenolanib or the salt thereof is at least one of crenolanib besylate, crenolanib phosphate, crenolanib lactate, crenolanib hydrochloride, crenolanib citrate, crenolanib acetate, crenolanib toluenesulphonate, and crenolanib succinate.

23. The method of claim 17, wherein the method further comprises at least one of:
providing crenolanib or the salt thereof up to three times or more a day for as long as the human patient is in need of treatment; or
providing crenolanib or the salt thereof at least one of sequentially or concomitantly, with another pharmaceutical agent in a newly diagnosed AML, to maintain remission, or in a relapsed/refractory AML; or
providing crenolanib or the salt thereof as a single agent or in combination with another pharmaceutical agent in a newly diagnosed AML, to maintain remission, or in a relapsed/refractor AML; or
providing crenolanib or the salt thereof as a single agent or in combination with another pharmaceutical agent in a newly diagnosed AML pediatric patient, to maintain remission, or in a relapsed/refractory AML pediatric patient.

24. The method of claim 17, wherein the human patient is relapsed/refractory to a prior tyrosine kinase inhibitor.
